(19) United States
(12) Patent Application Publication
(10) Pub. No.: US 2004/0152722 A1
(43) Pub. Date: Aug. 5, 2004

(54) Use of phencynonate hydrochloride

(76) Inventors: Chuangui Liu, Beijing (CN); Qishan Liang, Beijing (CN); Zhanguo Gao, Beijing (CN); Wenyu Cui, Beijing (CN)

Correspondence Address: HARNESS, DICKEY & PIERCE, P.L.C., P.O. BOX 828, BLOOMFIELD HILLS, MI, 48303, US

(21) Appl. No.: 10/469,395

(22) PCT Filed: Feb. 28, 2002
(86) PCT No.: PCT/CN02/00131

(30) Foreign Application Priority Data
Feb. 28, 2001 (CN) 01104881.6

(51) Int. Cl. A61K 31/46

(52) U.S. Cl. 514/304

(57) ABSTRACT

This invention relates to the new use of Phencynonate Hydrochloride in pharmaceutical field, especially its use for treating or alleviating Parkinson's disease or Parkinson's syndrome.

### TECHNICAL FIELD

This invention relates to the new use of Phencynonate Hydrochloride in the manufacture of medicament for treating or alleviating Parkinson's disease or Parkinson's syndrome.

### BACKGROUND ART

The structure of Phencynonate Hydrochloride, 2-phenyl-2-cyclopentyl-2-hydroxyacetic acid-3-methyl-3-azabicyclo(3, 3, 1) nonane-9 a-ester hydrochloride as the systematic name, is as follows:

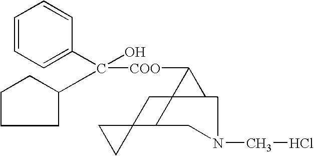

Chinese Patent applications No. 97125424.9 and No. 9311949491.1 disclosed the preparation method and its use as anti-motion sickness (such as car sickness, seasickness and airsickness, etc.).

Parkinson's disease is most commonly seen among elders, and the pathogenesis of this disease is still not clear. But evidences showed that degeneration of dopamine neuron in the patients' substantia nigra and striatum may result in hypofunction of dopamine system of the brain, as well as hyperfunction of cholinergic systern. Parkinson's disease is characterized by a series of symptoms of disturbance of extrapyramidal system, such as tremor, rigidity, akinesia, loss of postural reflex and the like. Once one catches the disease, he or she will suffer from its lifelong. Anticholinergic agents have been used for treating Parkinson's disease for 100 years. It was the only drug for Parkinson's disease treatment before 1970s. Presently, Benzhexol, Benzyltropine, Kemadrin, etc are commonly used anticholinergic agents in clinical practice in treating Parkinson's disease. They can effectively control the mild to moderate level symptoms during the early stage of Parkinson's disease. Though they are not stronger in potency than dopamine-agonists developed later, they do posses the advantages of less side effects in long term administration and good tolerance by patients. In recent years, more and more neurologists take central anticholinergics as their first choice on Parkinson's disease treatment during the early period, thus they can put off the prescription of dopamines, reduce the dosage of dopamines, consequently the intolerable side-effects of long term administration of dopamines are greatly alleviated and postponed. Parkinson's syndrome is resulted from hypofuntion of dopamine system and hyperfunction of cholinergic system in the brain, which are frequently caused by pharmaceutical, environmental factors or other nervous system diseases. It is also characterized by a series of signs of disturbance of extrapyramidal system as Parkinson's disease. If the cause is eliminated, then the disease can be cured. Tranquilizers administered by schizophrenia patients such as Phenothiazines (eg. Chlorpromazine), Thioxanthenes (eg. Chlorpyrifos) or Butyrophenones (eg. Haloperidol) which posses anti-dopamine action are the most important drugs among them.

Through competitive binding to DA-receptors in the striatum, these drugs can exert their therapeutic effects on patients with schizophrenia. However, at the same time, these drugs inevitably result in hyperfunction of cholinergic system and finally lead to a series of symptoms of disturbance of extrapyramidal system. It is much alike the pathogenesis of Parkinson's disease. In order to preserve these drugs' therapeutic effects and control their side effects at the same time, the only choice is to further administer CNS anticholinergic agents. These drugs are equal to those treating Parkinson's disease, such as Benzhexol, Benzyltropine and Kemadrin. When side effects of disturbance of extrapyramidal system due to administration of tranquilizers occur, combination with these drugs can control said side effects.

### OBJECT OF INVENTION

One object of this invention is to provide a medicament for treating or alleviating Parkinson's disease.

### BRIEF DESCRIPTION OF INVENTION

The inventors found that Phencynonate Hydrochloride can effectively alleviate the signs of Parkinson's disease or Parkinson's syndrome. It has lower ED₅₀ than known drugs that have been used in treating Parkinson's disease.

Therefore, this invention relates to the new use of Phencynonate Hydrochloride in the manufacture of medicament for treating or alleviating Parkinson's disease or Parkinson's syndrome.

This invention is also directed to a method of treating or alleviating Parkinson's disease or Parkinson's syndrome comprising administering effective amount of Phencynonate Hydrochloride to patient in need.

This invention also involves a pharmaceutical composition for treating or alleviating Parkinson's disease or Parkinson's syndrome comprising Phencynonate Hydrochloride and pharmaceutical vehicle and excipient.

### DETAILED DESCRIPTION OF THE INVENTION

The following examples were intended to illustrate the invention in detail without limiting the scope of the present invention in any way.

### EXAMPLE 1

### Antagonistic Action of Phencynonate Hydrochloride on Mice Rigor Model Induced by Haloperidol.

Haloperidol is a drug useful for schizophrenia treatment by blocking dopamine receptors in the brain, meanwhile, it can also cause disturbance of extrapyramidal system. This model is one of the accepted animal moedels for studies of Parkinson's disease and Parkinson's syndrome in the art.

Two hundred male mice, weighed 20-26 g, were used One hundred and twenty minutes after i.p. injection of haloperidol (5 mg/ml, diluted to 3.0 mg/kg/10 ml with 0.9% NaCl, available from HAI PU Pharmaceutical company, Shanghai), the forelimbs of a mouse was put on a stick of 0.9 cm in diameter, 100 cm in length and 3 cm in height. The researcher begin to time when the mouse lied in rigidity on the stick, and when both of the forelimbs of the mouse left the stick or the hindlimbs moved onto the stick, it was considered disappearance of rigidity and stopped timing. Thus the duration was considered as time of rigidity. Mice in the treatment group were administered Phencynonate Hydrochloride (5 dose levels: 1.0, 5.0, 10.0, 15.0 and 20.0 mg/kg/10 ml) intragastrically or positive control drug immediately after the administration of haloperidol.

Benzhexol (3 dose levels: 10.0, 20.0 and 30.0 mg/kg/10 ml) was administered intragastrically. As stated above, the forelimbs of the mouse were put on sticks, the time of rigidity is determined. Taking the average rigidity time of haloperidol model group as 100%, calculated the percentages of rigidity time of the two drugs at different dosage levels, as well as the dosage of the two drugs were calculated when rigidity time was shortened by 50%, i.e. ED₅₀ values (See Table 1).

From table 1, it can be seen that Phencynonate Hydrochloride has significant antagonistic action on this model, its ED₅₀ value is much lower than that of Benzhexol. Statistical analysis showed that the difference was significant (P<0.01).

TABLE 1
Phencynonate Hydrochloride's Antagonistic Action
on Rigidity Model of Mouse Induced By Haloperidol

| | Dose | Number | Rigidity | ED₅₀ + L₉₅ |
|---|---|---|---|---|
| | (mg/kg, oral) | of Animal | Incidence(%) | (mg/kg, oral) |
| Model of | Control | 20 | 100 | |
| Haloperidol | | | | |
| Phencynonate | 1.0 | 21 | 100 | 11.29 ± 1.75* |
| Hydrochloride | 5.0 | 10 | 65.06 | |
| | 10.0 | 19 | 54.16 | |
| | 15.0 | 8 | 30.97 | |
| | 20.0 | 18 | 28.29 | |
| Benzhexol | 10.0 | 19 | 94.03 | 19.56 ± 1.44 |
| | 20.0 | 22 | 47.96 | |
| | 30.0 | 22 | 16.05 | |

Comparison of Phencynonate Hydrochloride with Benzhexol, P < 0.01

### Example 2

### Antagonistic Action of Phencynonate Hydrochloride on Tremor Model of Mice Induced by Agonist of Cholinergic M-Receptor: Arecaline.

Tremor, as one of the main signs of Parkinson's disease and Parkinson's syndrome, may be induced by agonists of cholinergic M-receptor. It is also a recognized model of Parkinson's disease/Parkinson's syndrome in the art. 190 male mice, weighed 18-26 g, were injected with arecaline subcutaneously in dorsal area (Sigma, 8.0 mg/kg/10 ml). Count the number of mice developing tremor within 10 minutes after injection. Mice in the treatment group were given Phencynonate Hydrochloride (1.0, 1.8, 2.4, 3.0 mg/kg/10 ml) intragastrically or benzhexol (Sigma, 2.5, 5.0, 7.5, 10.0, 12.5 mg/kg/10 ml) intragastrically as positive control 45 minutes before administration of arecaline. Similarly, the number of mice developing tremor were counted Within 10 minutes after administration of arecaline, and the dose that decreased the incidence of tremor by 50%, i.e. ED₅₀ value was calcalated (See Table 2).

TABLE 2
Antagonistic Action of Phencynonate Hydrochloride
on Quiver Model of Mouse Induced by Arecaline

| | | Non-quiver | |
|---|---|---|---|
| | Dose | number/Total | ED₅₀ + L₉₅ |
| | (mg/kg, oral) | number | (mg/kg, oral) |
| Arecaline | Control | 0/10 | |
| Phencynonate | 1.0 | 3/20 | 2.05 ± 0.31* |
| Hydrochloride | 1.8 | 6/20 | |
| | 2.4 | 13/20 | |
| | 3.0 | 16/20 | |
| Benzhexol | 2.5 | 0/20 | 8.82 ± 0.83 |
| | 5.0 | 1/20 | |
| | 7.5 | 4/20 | |
| | 10.0 | 13/20 | |
| | 12.5 | 14/20 | |

Comparison of Phencynonate Hydrochloride with Benzhexol, P < 0.01

From table 2, it can be seen that Phencynonate Hydrochloride bad significant antagonistic action on this tremor model of mouse, its ED₅₀ value is much lower than that of benzhexol, statistical analysis showed significant difference, P<0.01.

The above two mice models proved that this invention had obvious effects of anti-Parkinson's disease and Parkinson's syndrome.

1. A use of Phencynonate Hydrochloride of formula in the manufacture of medicament for treating or alleviating Parkinson's disease or Parkinson's syndrome. [chemical structure]
